(19) United States
(12) Patent Application Publication
(10) Pub. No.: US 2007/0249916 A1
(43) Pub. Date: Oct. 25, 2007

(54) Wearable Glucometer

(75) Inventors: Benny Pesach, Rosh- Ha'ayin (IL); Ron Nagar, Tel-Aviv (IL); Gabriel Bitton, Jerusalem (IL); Avner Adoram, Ramat-Hasharon (IL)

Correspondence Address: WOLF, BLOCK, SCHORR & SOLIS-COHEN LLP, 250 PARK AVENUE, NEW YORK, NY, 10177, US

(73) Assignee: Glucon, Inc., Boulder, CO (US)

(21) Appl. No.: 10/597,108

(22) PCT Filed: Jan. 15, 2005
(86) PCT No.: PCT/IL05/00046

§ 371 Date: Jul. 3, 2007

Related U.S. Application Data
(60) Provisional application No. 60/536,510, filed on Jan. 15, 2004.

(51) Int. Cl. A61B 5/00

(52) U.S. Cl. 600/316; 600/322

(57) ABSTRACT

Apparatus for assaying an analyte in blood in a patient's blood vessel comprising: a light provider comprising at least one light source that illuminates a tissue region in which a blood vessel is located with light that stimulates photoacoustic waves in the region; at least one acoustic transducer that generates signals responsive to the photoacoustic waves; a controller that receives the signals and processes them to determine which are responsive to photoacoustic waves that originate in the blood vessel and uses the determined signals to assay the analyte; wherein, the light provider and at least one transducer define a field of view that overlaps the blood vessel, said field of view having a central region and a lateral extent greater than about 4 mm.

### RELATED APPLICATIONS

The present application claims the benefit under 35 USC 119(e) of U.S. provisional application No. 60/536,510 filed on Jan. 15, 2004, the disclosure of which is incorporated herein by reference.

### FIELD OF THE INVENTION

The invention relates to wearable apparatus that can be coupled to a body and continuously assay a substance in the body for an extended period of time and in particular wearable apparatus for continuously monitoring glucose levels in a body.

### BACKGROUND OF THE INVENTION

Methods and apparatus for determining blood glucose levels for use in the home, for example by a diabetic who must monitor blood glucose levels frequently, are available. These methods and associated devices are generally invasive and usually involve taking blood samples by finger pricking. Often a diabetic must determine blood glucose levels many times daily and finger pricking is perceived as inconvenient and unpleasant. To avoid finger pricking, diabetics tend to monitor their glucose levels less frequently than is advisable.

Non-invasive in-vivo methods and apparatus for monitoring blood glucose are known. PCT Publication WO 98/38904, the disclosure of which is incorporated herein by reference, describes a “non-invasive, in-vivo glucometer” that uses a photoacoustic effect to measure a person's blood glucose. PCT Publication WO 02/15776, the disclosure of which is incorporated herein by reference, describes locating a blood vessel in the body and determining glucose concentration in a bolus of blood in the blood vessel. The glucose concentration in the blood bolus is determined by illuminating the bolus with light that is absorbed and/or scattered by glucose to generate photoacoustic waves in the bolus. Intensity of the photoacoustic waves, which is a function of glucose concentration, is sensed and used to assay glucose in the bolus.

Wearable devices for assaying glucose are known, are generally based on near-infrared (NIR) spectroscopic methods and usually comprise a light source and optical detector that are attached to a patient's finger, wrist or other part of the body. Wearable NIR devices for assaying glucose are described in U.S. Pat. No. 6,241,663 to Wu, et al. and U.S. Pat. No. 5,551,422, to Simonsen et al., the disclosures of which are incorporated herein by reference.

An apparatus for determining glucose levels is hereinafter referred to as a “glucometer”.

### SUMMARY OF THE INVENTION

An aspect of some embodiments of the present invention relates to providing a wearable glucometer that may be mounted to a patient's skin in alignment with a blood vessel in the patient's body and thereafter operates to repeatedly assay glucose in blood in the blood vessel without requiring substantial user intervention.

It is generally advantageous to determine glucose levels for a patient from blood glucose levels. Prior art wearable glucometers do not in general distinguish between glucose levels in blood and glucose levels in interstitial fluid and cannot therefore assure that glucose assays they provide are blood glucose levels. Unlike prior art wearable glucometers, a glucometer in accordance with an embodiment of the invention provides measurements of glucose levels that are substantially independent of glucose levels in interstitial fluid.

An aspect of some embodiments of the present invention relates to providing a glucometer, which once aligned with a blood vessel will continue to operate properly, providing glucose assays for blood in the blood vessel, in the event that it becomes misaligned by displacements typically encountered during assay operation.

A glucometer in accordance with an embodiment of the present invention comprises an array of acoustic transducers, a light provider, and a controller. The controller controls the light source and the array of transducers to assay glucose in blood in the patient's blood vessel using a photoacoustic effect. To perform the assay, the controller controls the light provider to illuminate a tissue volume defined by a field of view of the glucometer located below the skin to which the glucometer is attached with light that is absorbed and/or scattered by glucose and stimulates photoacoustic waves in the tissue volume. The field of view of the glucometer is defined as a size and location of a volume of tissue below a region of skin to which the glucometer is attached for which the glucometer stimulates photoacoustic waves that are detectable by its transducer array and practically useable to assay glucose in blood in a blood vessel located in the tissue volume. When properly aligned with the blood vessel, a region of the blood vessel is located substantially at the center of the glucometer's field of view. The transducer array generates signals responsive to acoustic energy that is incident on the array from the photoacoustic waves stimulated in the tissue volume.

The controller receives and processes the signals provided by the transducer array to determine which of the signals corresponds to photoacoustic waves originating in the blood vessel and uses those signals in accordance with methods known in the art to assay glucose in blood in the blood vessel. Examples of photoacoustic assay methods useable in the practice of the invention are described in PCT publication WO 02/15776, and in U.S. Provisional Application 60/458,973 filed on Apr. 1, 2003, the disclosures of which are incorporated herein by reference.

In time, during extended assay operation, a glucometer initially properly aligned with a blood vessel so that a region of the blood vessel is located at the center of the glucometer's field of view, may become misaligned because, for example, of drift in the glucometer position on the skin or because of motion of the skin relative to the blood vessel.

In accordance with an aspect of an embodiment of the invention, the transducer array and light provider are configured so that the field of view of the glucometer is sufficiently large in at least one dimension so that for misalignments typically encountered during assay operation, the blood vessel remains substantially within the glucometer field of view. As a result, assay operation can continue satisfactorily uninterrupted.

In some embodiments of the invention, to align the glucometer with a blood vessel the controller controls the array of transducers to acoustically image a tissue region in the patient's body beneath the skin. In some embodiments of the invention, to align the glucometer, the controller controls the light provider to illuminate the field of view of the glucometer with light that stimulates photoacoustic waves in the glucometer field of view. The controller processes signals generated by the transducer array responsive to the photoacoustic waves to generate a “photoacoustic image” of features below the skin.

The acoustic and/or photoacoustic image provided by the controller is used to align the glucometer with the blood vessel. Optionally, the controller generates a signal responsive to the acoustic and/or photoacoustic image to aid a user of the glucometer to align the glucometer with the blood vessel. Optionally, the glucometer comprises a display screen and the controller displays the acoustic and/or photoacoustic image, or icons responsive to the images, to facilitate aligning the glucometer with the blood vessel.

In some embodiments of the invention, the glucometer is coupled to an insulin pump which is mounted to the patient. The glucometer controls the insulin pump to administer insulin to the patient responsive to glucose measurements acquired by the glucometer.

There is therefore provided, in accordance with an embodiment of the invention, apparatus for assaying an analyte in blood in a patient's blood vessel comprising: a light provider comprising at least one light source that illuminates a tissue region in which a blood vessel is located with light that stimulates photoacoustic waves in the region; at least one acoustic transducer that generates signals responsive to the photoacoustic waves; a controller that receives the signals and processes them to determine which are responsive to photoacoustic waves that originate in the blood vessel and uses the determined signals to assay the analyte; wherein, the light provider and at least one transducer define a field of view that overlaps the blood vessel, said field of view having a central region and a lateral extent greater than about 4 mm.

Optionally, the field of view has a lateral extent greater than or equal to about 6 mm. Optionally, the field of view has a lateral extent greater than or equal to about 10 mm.

In some embodiments of the invention, the light provider comprises optics for each of the at least one light source that receives light from the light source and configures the received light into a fan shaped light beam that is used to illuminate the tissue region.

Optionally, the at least one light source comprises a plurality of light sources. Optionally, the fan beams of the plurality of light sources are substantially parallel. Optionally, the plurality of light sources are collinear. Optionally, the plurality of light sources are configured in an array of rows and columns.

In some embodiments of the invention, the light provider comprises a mirror that receives light from the light source and reflects the received light to the tissue region and wherein the mirror is rotatable about an axis and for different rotation angles of the mirror about the axis the fan beam illuminates a different portion of the tissue region. Optionally the apparatus comprises a controller that controls the angle of the mirror to scan the tissue region with light from the light source.

In some embodiments of the invention, the light provider comprises a light pipe having an input surface region to which at least one light source is coupled and an output surface region through which light that enters the light pipe from the at least one light source exits the light pipe. Optionally, the light pipe has a shape of a planar plate having two large parallel face surfaces and narrow edge surfaces, Optionally, the input surface region to which the at least one light source is coupled is a narrow edge surface of the light pipe. Optionally, the output surface region from which light exits the light pipe is a narrow edge surface opposite the input surface region.

In some embodiments of the invention, the at least one transducer comprises a plurality of transducers. Optionally, the transducers are configured in an array of rows and columns of transducers. Additionally or alternatively, the apparatus comprises a mounting plate, which is attached to the skin to acoustically couple the apparatus to the skin. Optionally, the transducers are mounted to the mounting plate. Optionally, the mounting plate comprises a layer of piezoelectric material. Optionally, each of at least two of the plurality of transducers comprises a different region of the layer of piezoelectric material sandwiched between a first and a second electrode. Optionally, the first electrodes of each of the at least two transducers are substantially electrically isolated from each other. Optionally, the second electrode of each of the at least two transducers comprises a different region of a same conductor.

In some embodiments of the invention, a transducer of the at least one transducer is acoustically coupled to the skin via an acoustic waveguide. Optionally, the acoustic waveguide is an optic fiber.

In some embodiments of the invention, a light source of the at least one light source is optically coupled to the skin via an optic fiber that transmits light from the light source to the skin. Optionally, a transducer of the at least one transducer light is acoustically coupled to the skin by the optic fiber.

In some embodiments of the invention, the controller controls the at least one transducer to acoustically image the blood vessel.

In some embodiments of the invention, the controller processes signals generated by the at least one transducer responsive to acoustic energy from the photoacoustic waves to image the blood vessel. Optionally, at least some of the light provided by the light provider is light at a wavelength at which light is strongly absorbed and or scattered by blood.

In some embodiments of the invention, the controller uses the image to determine if the blood vessel is substantially aligned with the central region of the field of view. Optionally, the apparatus comprises an indicator light and the controller controls the indicator light to generate an optical signal indicative of a degree to which the blood vessel is aligned with the central region. Additionally or alternatively, the apparatus comprises a speaker and the controller controls the speaker to generate an audio signal indicative of a degree to which the blood vessel is aligned with the central region.

In some embodiments of the invention, the apparatus comprises a display screen and the controller displays a fiducial mark representing the central region of the field of view and the image of the blood vessel on the screen and wherein a distance on the screen between the blood vessel and the fiducial mark represents a distance between the blood vessel and the central region.

In some embodiments of the invention, the light provider and at least one transducer are comprised in a wearable housing. Optionally, when worn by the patient the housing provides optical and acoustic coupling of the light provider and at least one transducer respectively to the patient's skin.

In some embodiments of the invention, the analyte is glucose.

There is further provided in accordance with an embodiment of the invention apparatus for controlling blood glucose level in a patient comprising: assay apparatus according to an embodiment of the invention; an insulin delivery system controllable to administer insulin to a patient; wherein the controller controls the insulin delivery system responsive to glucose assays provided by the assay apparatus.

### BRIEF DESCRIPTION OF FIGURES

Non-limiting examples of embodiments of the present invention are described below with reference to figures attached hereto, which are listed following this paragraph. In the figures, identical structures, elements or parts that appear in more than one figure are generally labeled with a same numeral in all the figures in which they appear. Dimensions of components and features shown in the figures are chosen for convenience and clarity of presentation and are not necessarily shown to scale.

FIGS. 1A and 1B schematically show a perspective view and cross-section view respectively of a glucometer, in accordance with an embodiment of the present invention;

FIGS. 2A and 2B schematically show a perspective view and cross-section view respectively of a glucometer comprising a linear array of light sources, in accordance with an embodiment of the present invention;

FIG. 2C schematically shows a perspective view of a glucometer comprising a two dimensional array of light sources, in accordance with an embodiment of the present invention;

FIG. 2D schematically shows a perspective view of another glucometer, in accordance with an embodiment of the present invention;

FIGS. 3A and 3B schematically show a perspective view and cross-section view respectively of a glucometer having a light beam that can be steered to scan tissue below a region of skin to which the glucometer is mounted, in accordance with an embodiment of the present invention;

FIG. 3C schematically shows another glucometer having a steerable light beam, which is similar to the glucometer shown in FIGS. 3A and 3B, in accordance with an embodiment of the present invention; and

FIG. 4 schematically shows a glucometer comprising an array of light pipes for directing light to illuminate tissue below a region of skin to which the glucometer is mounted, in accordance with an embodiment of the present invention.

### DETAILED DESCRIPTION OF EXEMPLARY EMBODIMENTS

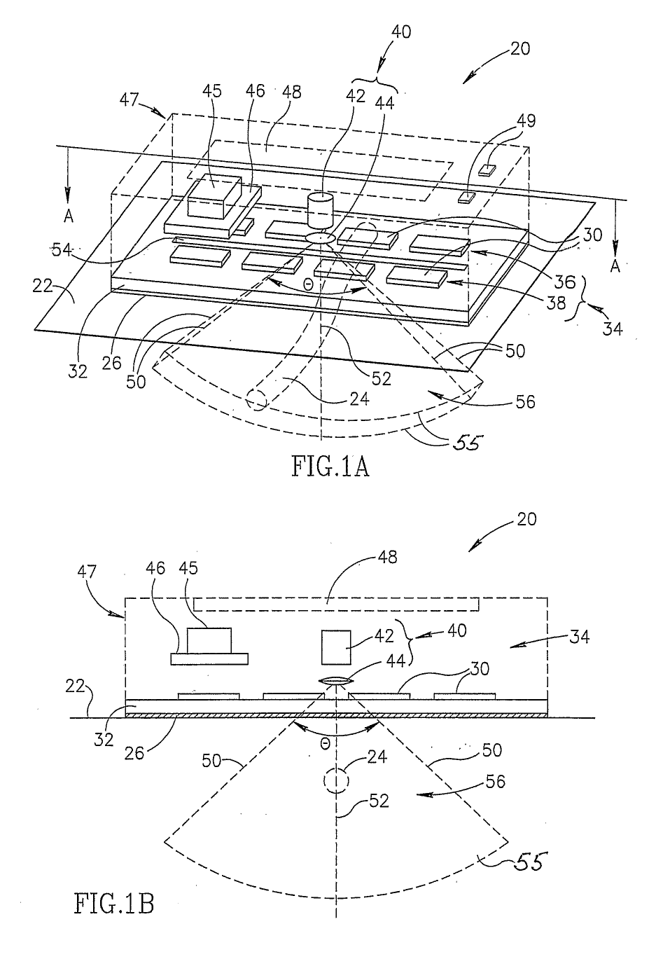

FIGS. 1A and 1B schematically show a perspective view and cross-section view respectively of a glucometer 20 in accordance with an embodiment of the present invention. The cross section view shown in FIG. 1B is taken in a plane indicated by a line “AA” in FIG. 1A. Glucometer 20 is shown attached to a region of skin 22 of a patient after it has been aligned with a blood vessel 24 located under the patient's skin in order to assay glucose in blood in the blood vessel.

Glucometer 20 comprises a plurality of acoustic transducers 30 mounted to a mounting plate 32, which are optionally configured in an array 34 of rows 36 and columns 38, and a light provider 40 comprising a light source 42 and optics represented by a lens 44. By way of example, the number of transducers 30 in array 34 is eight and the number of rows 36 and columns 38 in the array are two and four respectively. A controller 46 controls light provider 40 and transducer array 34. The components of glucometer 20 are comprised in a housing 47 indicated by dashed lines.

Optionally, a power source 45 for powering controller 46 and light source 42 is mounted inside housing 47. In some embodiments of the invention, power for controller 46 and light source 42 is provided by an external power source to which glucometer 20 is connected. Optionally, the external power source is mounted to the patient's body. Housing 47 optionally has a visual display screen 48 and control buttons 49 for interfacing with controller 46. Glucometer 20 is optionally attached to skin 22 by a layer 26 of a suitable adhesive that bonds mounting plate 32 to skin 22.

In some embodiments of the invention, mounting plate 32 is formed from a flexible piezoelectric material, such as PVDF and acoustic transducers 30 are integrally formed elements of the mounting plate. Each integrally formed acoustic transducer 30 comprises a region of mounting plate 32 sandwiched between a first electrode on a top surface of the mounting plate and a second electrode on a bottom surface of the mounting plate. Voltage generated between the first and second electrode of a transducer 30 responsive to acoustic energy incident on the transducer is used to sense the acoustic energy. Whereas first electrodes of transducers are substantially electrically isolated from each other, each second electrode may be a region of a same single large electrode optionally on the bottom surface of the mounting plate.

In some embodiments of the invention, mounting plate 32 comprises a flexible membrane, which is adhered to the skin by a suitable adhesive, and each transducer 30 comprises a reflective coating on a different region of the membrane, which may be a different region of a single continuous reflective coating on the membrane. A suitable light source is used to scan and selectively illuminate the reflective coatings. Light from the light source reflected by a given reflective coating is received by an optical sensor or sensor system. Acoustic energy incident on the membrane distorts and/or displaces regions of the membrane and thereby distorts and/or displaces reflective coatings on the membrane. Intensity and/or phase of light from the light source reflected by a reflective coating of a given transducer and/or a location on the sensor or sensor system at which the reflected light is received is responsive to the distortion and/or displacement and is used to generate a signal responsive to the incident acoustic energy. Methods of sensing acoustic energy responsive to intensity, phase or location of incidence on an optical detector of light reflected from a flexible membrane on which the acoustic energy is incident are known in the art and any of these methods may be used in the practice of the present invention.

For convenience of presentation, in FIGS. 1A and 1B and figures that follow transducers 30 are shown as separate elements mounted on mounting plate 32.

Light from light source 42 is optionally shaped by optics 44 into a relatively thin fan shaped beam of light schematically indicated by dashed lines 50 and directed so that it is incident on mounting plate 32 between rows 36 of transducers 30. Fan beam 50 has a central axis 52 and a fan angle θ. To enable light in fan beam 50 to pass through mounting plate 32 and illuminate tissue below skin 22, mounting plate 32 is optionally formed from a material that is transparent to light in fan beam 50. Additionally or alternatively, mounting plate 32 is formed with a slot 54 through which light beam 50 passes. Optionally, adhesive layer 26 is formed from a material that is transparent to light in fan beam 50. Additionally or alternatively, adhesive layer 26 does not cover slot 54 so as not to interfere with passage of light through the slot.

Intensity of light in fan beam 50 and a number and configuration of transducers 30 in array 34 are such that photoacoustic waves stimulated by the light beam in tissue to a depth below skin 22 indicated by dashed “depth” lines 55 are generally detectable by the transducer array. A region 56 of the tissue in which photoacoustic waves that are detectable by transducer array 34 are stimulated is substantially bounded by the envelope of fan beam 50 and dashed depth lines 55. Region 56 is coincident with the field of view of glucometer 20 and will be referred to as “field of view 56”.

Since glucometer 20 is assumed to be aligned with blood vessel 24, the blood vessel passes substantially through axis 52 in a direction substantially perpendicular to the plane of fan beam 50. In accordance with an embodiment of the invention, lens 44 forms fan beam 50 having a fan angle θ large enough so that at an expected depth of blood vessel 24 below skin 22 a cross section of field of view 56 in the plane of the fan beam 50 is substantially larger than a typical cross section of the blood vessel.

Optionally, fan beam 50 is configured so that at a depth of blood vessel 24 below skin 22, fan beam 50 extends on either side of the blood vessel by a distance, hereinafter an “alignment margin”, equal to about 3 mm. For example, for a diameter of blood vessel 24 equal to about 1 mm and having an expected location about 2 mm below the surface of skin 22, fan beam 50 is optionally configured so that at about 2 mm below the skin, width of the fan beam in the plane of the fan beam is equal to or greater than about 7 mm and the fan beam has a fan angle θ equal to about 120°.

In some embodiments of the invention, a glucometer similar to glucometer 20 is configured to have an alignment margin different from about 3 mm. For example, for a glucometer similar to glucometer 20 that is to be used to monitor glucose levels in an athlete during exercise, displacements by which the glucometer might become misaligned may be expected to be greater than usual and the glucometer configured to have an alignment margin greater than about 3 mm. Optionally the alignment margin is equal to about 5 mm. For a bed-ridden patient a glucometer may have an alignment margin less than about 3 mm. Optionally, the alignment margin is equal to about 2 mm.

To align glucometer 20 with blood vessel 24 as shown in FIGS. 1A and 1B, glucometer 20 is placed on a region of skin 22 below which blood vessel 24 is expected to be located. A suitable gel or oil is optionally used to acoustically couple the glucometer to the skin. A control signal is input to glucometer 20 via interface buttons 49 instructing controller 46 to operate in an alignment mode and the glucometer is oriented so that the plane of fan beam 50 is substantially perpendicular to the length of the blood vessel. Optionally, controller 46 indicates orientation of the plane of fan beam 50 by generating a suitable icon on display screen 48.

The patient and/or a person aiding the patient, then moves glucometer 20 back and forth substantially in a direction perpendicular to the length of blood vessel 24. Optionally, during motion of glucometer 20, controller 46 controls transducer array 34 to image features below skin 22 and in particular blood vessel 24 with ultrasound using methods known in the art. In some embodiments of the invention, Doppler shifted ultrasound imaging methods known in the art are used to image blood vessel 24. Optionally, during motion of glucometer 20, controller 46 controls light provider 40 to illuminate tissue below skin 22 with light that stimulates photoacoustic waves in the tissue. Optionally, controller 467 controls light provider 40 to illuminate tissue below skin 22 with light at at least one wavelength that is strongly absorbed by blood. Signals generated by transducer array 34 responsive to the photoacoustic waves are used to provide a “photoacoustic” image of features below skin 22 and in particular blood vessel 24.

Optionally, controller 46 generates a signal responsive to the ultrasound and/or photoacoustic image to aid a user of glucometer 20 to align the glucometer with the blood vessel. For example, controller 46 may control a LED and/or a small speaker (not shown) responsive to the image to provide an optical and/or audio signal indicating when glucometer 20 is aligned with blood vessel 24.

Optionally, controller 46 displays the ultrasound and/or photoacoustic image on screen 48 to facilitate aligning the glucometer with the blood vessel. For example, in some embodiments of the invention controller 46 displays the ultrasound or photoacoustic image on screen 48 together with a suitable fiducial mark representing the center of the field of view of glucometer 20. The patient, and/or the patient's aid, aligns glucometer 20 with blood vessel 24 responsive to a location in the image of blood vessel 24 relative to the fiducial mark.

Once the glucometer is substantially aligned with blood vessel 24, the position of the aligned glucometer on the patient's skin is optionally marked using any suitable marking device, such as a pen for marking skin with non-toxic ink. The patient then removes glucometer 20 from skin 22 and applies a layer of adhesive 26 to mounting plate 32 or removes a protective coating on a layer of adhesive 26 already in place on the mounting plate. The patient and/or the patient's aid then repositions glucometer 20 on skin 22 responsive to the alignment marks with the adhesive in contact with the skin and presses the glucometer to the skin to assure proper contact of the skin to the adhesive. Methods of aligning a glucometer with a blood vessel are described in U.S. Provisional Application 60/476,623, filed on Jun. 9, 2003, the disclosure of which is incorporated herein by reference.

Once properly aligned, a control signal is input to the glucometer via interface buttons 49 instructing controller 46 to operate in an assay mode to assay glucose in blood vessel 24. In the assay mode controller 46 controls light provider 40 to illuminate region 56 with fan beam 50 at at least one wavelength that is scattered and/or absorbed by glucose. Signals generated responsive to photoacoustic waves generated in blood in blood vessel 24 by the light are used to determine concentration of glucose in the blood. Any suitable method known in the art for processing the signals to determine the glucose concentration in the blood may be used. As noted above, exemplary methods for assaying glucose in blood in blood vessel 24 responsive to a photoacoustic effect are described in PCT publication WO 02/15776 and in U.S. Provisional Application 60/458,973 cited above.

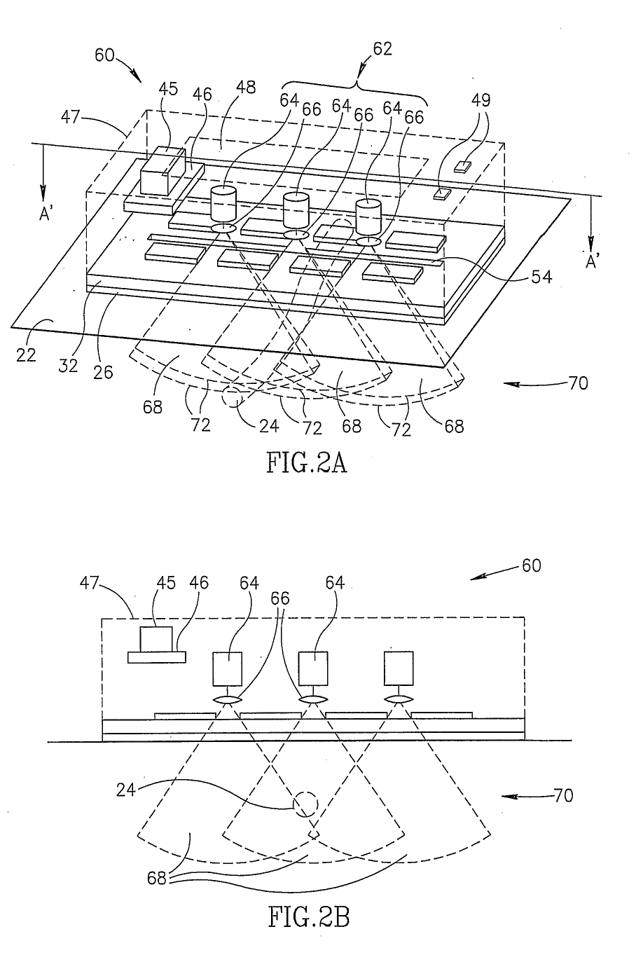

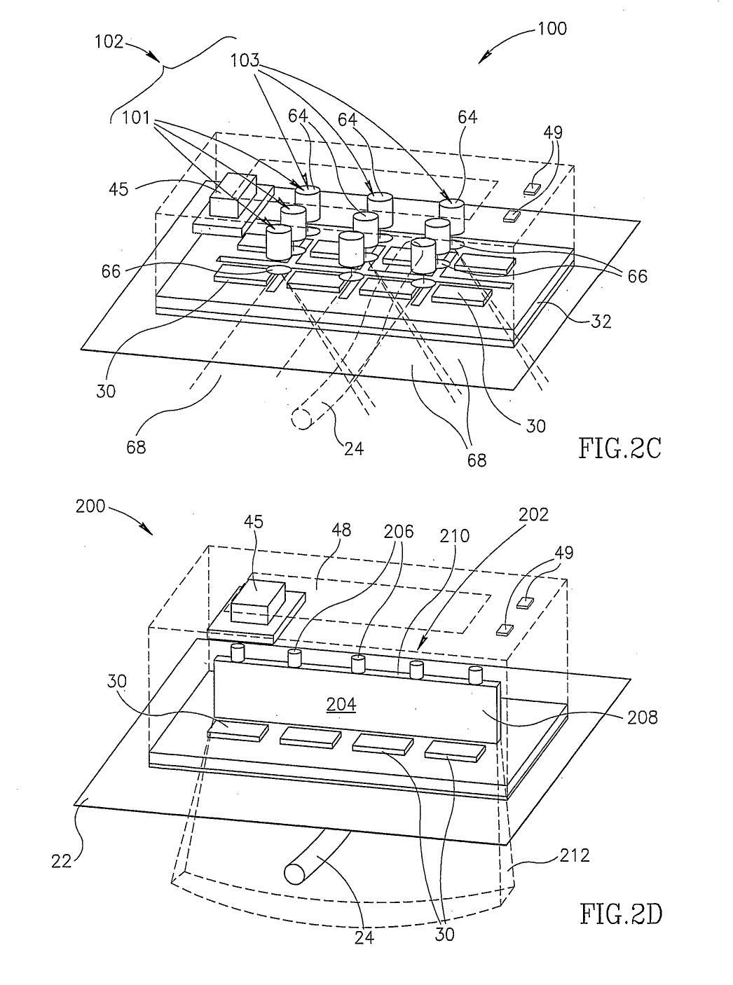

As a result of the relatively large fan angle θ of fan beam 50 and its orientation substantially perpendicular to blood vessel 24, even if glucometer 20 becomes substantially misaligned with the blood vessel, the blood vessel will in general remain inside field of view 56 of the glucometer. (Displacements of glucometer 20 in a direction along the length of blood vessel 24 do not in general result in the blood vessel being displaced relative to the center of the field of view of the glucometer. On the other hand, displacements in a direction perpendicular to the length of blood vessel 24, do in general result in the blood vessel displacing relative to the center of field of view 56. However, because of the relatively large opening angle θ of fan beam 50, for typical misaligning displacements of glucometer 20 perpendicular to the length of blood vessel 24, in general the blood vessel remains within field of view 56 of the glucometer.) As a result, degrees of misalignment typically encountered during operation of glucometer 20 will not in general substantially compromise satisfactory operation of the glucometer. It is expected that, for normal activity not including extreme physical exercise, glucometer 20 may become misaligned relative to blood vessel 24 during assay operation over a period of time equal to about a working day by distances of magnitude less than or equal to about 2 mm. FIGS. 2A and 2B schematically show a perspective view and a cross section view of another glucometer 60, in accordance with an embodiment of the present invention.

Glucometer 60 is similar to glucometer 20 except that glucometer 60 comprises a light provider 62 having a plurality of light sources 64 each optionally optically coupled to optics represented by a lens 66. By way of example, in FIGS. 2A and 2B the number of light sources 64 and associated optics 66 is equal to three. Light from each light source 64 is optionally formed by optics 66 associated with the light source into a fan beam of light 68. Light from each fan beam 68 passes through slot 54 to illuminate tissue beneath skin 22. The plurality of fan beams 68 provides glucometer 60 with a relatively large field of view 70 which is determined substantially by the envelopes of fan beams 68 and depth lines 72. Because a plurality of light sources 64 is used to provide field of view 70, light sources 64 may provide light at lower intensity than is provided by single light source 42 comprised in glucometer 20 (FIGS. 1A and 1B).

In some embodiments of the present invention, controller 46 controls light provider 62 so that less than all the light sources 64 are on simultaneously. By turning on less than all light sources 64 at a same given time, light from light provider 62 illuminates a known region of field of view 70, which is smaller than the field of view. At the given time therefore, photoacoustic waves stimulated by the light have origins in a spatial region smaller than that occupied by the field of view 70 and spatial resolution with which the origins can be located may be improved.

FIG. 2C schematically shows a glucometer 100 that is a variation of glucometer 60. Whereas glucometer 60 comprises a linear array of light sources 64, glucometer 100 comprises a two-dimensional array 102 of rows 101 and columns 103 of light sources 64. As in the case of glucometer 60, light from each light source 64 is optionally shaped by associated optics 66 into a fan beam 68 of light. The planes of fan beams 68 are optionally substantially parallel to each other. Fan beams 68 are shown for only a few light sources 64 for clarity of presentation and to prevent clutter. Optionally, mounting plate 32 is transparent to light in fan beams 68 and light in a fan beam 68 passes through the mounting plate to illuminate tissue below a region of a patient's skin 22 to which glucometer 100 is attached. Optionally, light from each light source 64 is transmitted through a suitably shaped slot 104 formed in mounting plate 32. In some embodiments of the invention, different rows 101 provide light at different wavelengths of a plurality of wavelengths used to assay glucose in blood vessel 24.

Glucometers 20 (FIGS. 1A and 1B) and 60 (FIGS. 2A and 2B) described above have fields of view that are relatively “thin” in directions perpendicular to their respective fan beams and relatively large in a direction parallel to the planes of their fan beams. As a result of the shape of their fields of view, it is generally advantageous to align glucometers 20 and 60 with a blood vessel so that the planes of their fan beams are substantially perpendicular to the length of the blood vessel. For “perpendicular alignment”, displacement of glucometer 20 or 60 along the length of blood vessel 24 does not substantially misalign the glucometers nor as a result substantially affect their operation. Displacement of glucometer 20 or 60 perpendicular to blood vessel 24 will generally not remove the blood vessel from their respective fields of view, and as a result will also not in general injure operation of the glucometers. Such perpendicular alignment is relatively easy to achieve for blood vessels in the arm or wrist whose lengths are often substantially parallel to the lengths of the appendages in which they are located.

Glucometer 100 shown in FIG. 2C has a field of view that is thicker in a direction perpendicular to the planes of its fan beams 68 than that of glucometers 20 and 60. As result of its thicker field of view, glucometer 100 may often be easier to align with a patient's blood vessel than are glucometers 20 and 60. It is expected that aligning glucometer 100 should be easier than aligning glucometers 20 and 60 with a blood vessel for situations in which a direction of a length of the blood vessel is not known or the patient's blood vessel is relatively twisted.

In glucometers 20, 60 and 100, a relatively large, in at least one direction, field of view is generated using, in addition to an appropriate array of transducers, optics to form at least one fan beam of light from light received from a suitable light source. In some embodiments of the invention, a relatively large field of view is provided using an array of acoustic transducers and a light pipe that receives light from at least one light source and transmits the light from an output aperture of the light pipe as a beam of light having a large cross section in at least one direction. For example, the light pipe may receive light from a plurality of light sources and transmit the received light from a relatively long output aperture to provide a beam of light having a large cross section.

FIG. 2D schematically shows an exemplary glucometer 200 comprising a light provider 202 comprising a light pipe 204 coupled to a plurality of light sources 206, in accordance with an embodiment of the present invention. Glucometer 200 optionally comprises an array of transducers 30 similar to that shown in FIGS. 1A, 1B and 2C. (Transducers 30 behind light pipe 204 are not shown.)

Light pipe 202 is optionally rectangular having relatively large face surfaces 208 and a relatively narrow edge surface 210 along which light sources 206 are coupled using methods known in the art. Optionally, substantially all surface regions of light pipe 204, except for a narrow edge surface, an “output aperture” of the light pipe, opposite edge surface 210, are covered with a reflective coating that reflects light provided by light sources 206. Light provided by light sources 206 exits light pipe 204 from the output aperture edge opposite edge 210 as a relatively thin but wide beam of light 212 that has a cross section in the plane of light pipe substantially larger than the cross section of blood vessel 24. Optionally, light pipe 204 is formed having scattering centers using methods known in the art to homogenize light that the light pipe receives from light sources 206 so that light exiting the light pipe has fairly uniform intensity along the output aperture.

It is noted that whereas light pipe 204 is rectangular, light pipes having shapes different from light pipe 204 and configurations of light sources different from that shown in FIG. 2D may be used in the practice of the present invention. For example, the light pipe may be a relatively long tube-like light pipe having a square, triangular or hemispherical cross section. The light pipe has a relatively long narrow surface running substantially the length of the tube that functions as an output aperture. Light is inserted into the light pipe from a light source optically coupled to a surface region at at least one end of the tube. In addition, more than one light pipe may be used to provide an appropriate light beam form illuminating a field of view of a glucometer, in accordance with an embodiment of the present invention.

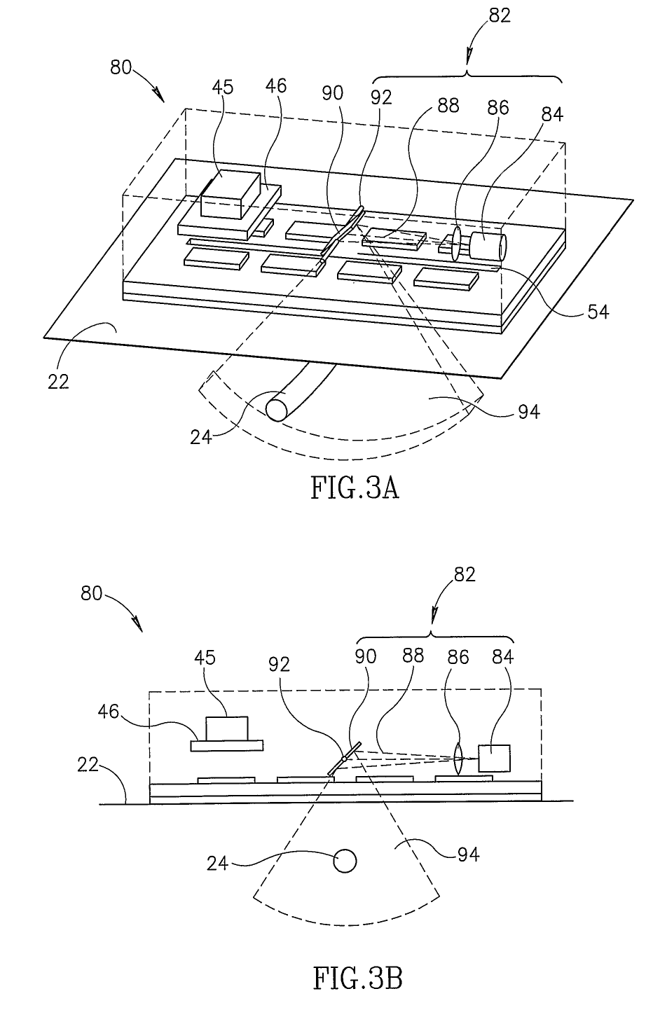

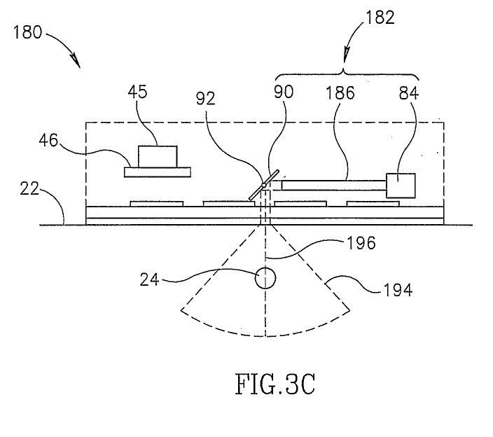

FIGS. 3A and 3B schematically show yet another glucometer 80, in accordance with an embodiment of the present invention. Glucometer 80 is similar to glucometers 20, 60 and 100 but comprises a light provider 82 having a light source 84 and associated optics 86 and a mirror 90. Optics 86 optionally forms light from light source 84 into a fan shaped beam 88 and directs the light to mirror 90, which is rotatable about an axis 92. Mirror 90 reflects light that it receives as a fan beam 94 through slot 54 to illuminate tissue below skin 22. Orientation of mirror 90 about axis 92 is controlled by controller 46 to direct fan beam 94 at different angles into tissue below skin 22 so that the light beam scans a relatively large region of the tissue and thereby provides glucometer 80 with a relatively large field of view.

In some embodiments of the invention, controller 46 correlates position of mirror 90 and thereby the position of fan beam 94 with acoustic signals generated by transducers 30 responsive to photoacoustic waves sensed by the transducers. By correlating the position of fan beam 94 with the acoustic signals, a portion of the field of the view of glucometer 80 in which the sensed photoacoustic waves originate is localized and spatial resolution for locating origins of the photoacoustic waves may be improved.

In the glucometers described above (glucometers 20, 60, 80 and 100), optics are optionally used to form light from a light source into a fan shaped light beam having a relatively large cross section in the plane of the fan beam. In accordance with some embodiments of the invention, scattering of light in body tissue is relied upon to provide a glucometer with a light beam having a relatively large cross section. Light from a light source in the glucometer is directed into body tissue at a localized spot on a skin region to which the glucometer is attached. Upon entry into the body tissue the tissue scatters the light and spreads it into a substantially cone shaped beam having a relatively large cross section in a plane through an axis of the cone.

FIG. 3C schematically shows a cross section view of a glucometer 180 similar to glucometer 80 comprising a light provider 182 having an optic fiber 186 to direct light from light source 84 to mirror 90, in accordance with an embodiment of the invention. Glucometer 180, optionally, does not comprise optics to shape light from light source 84 into a fan shaped light beam. Light from optic fiber 186 is reflected by mirror 90 onto skin 22. Upon entering tissue below skin the tissue scatters the light into a cone shaped beam 194 having a relatively large cross section in a plane that includes an axis 196 of cone beam 194, which plane in the cross section view of FIG. 3C is the plane of the paper.

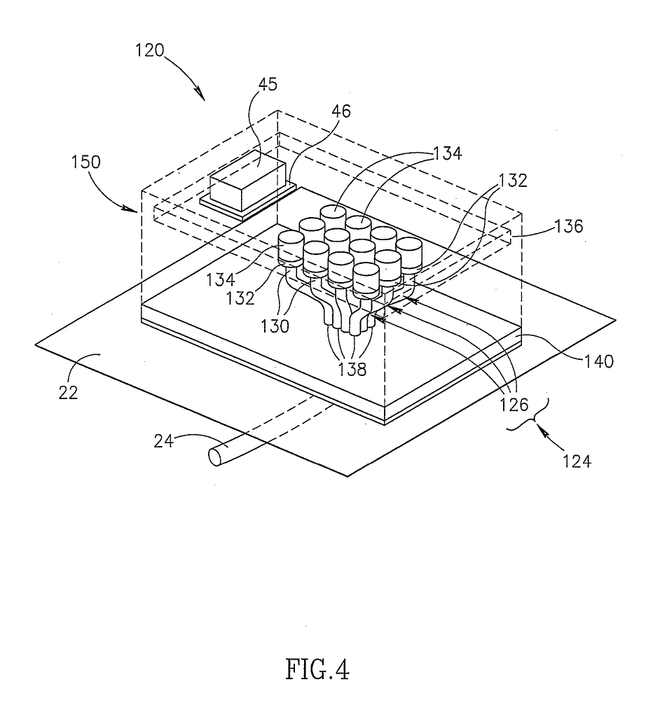

FIG. 4 schematically shows a glucometer 120 comprising a light provider having an array 124 of optic fibers 126 through which light is transmitted to illuminate tissue and blood vessel 24 below a region of a patient's skin 22 to which the glucometer is attached.

A first end 130 of each optic fiber 126 is optionally coupled to an acoustic transducer 132 and a suitable light source 134. Acoustic transducer 132 is formed from a piezoelectric material transparent to light provided by light source 134 and light radiated by the light source propagates through the piezoelectric sensor to enter optic fiber 126. Optionally, sources 134 and acoustic transducers 132 are formed in a suitable substrate 136 using micro-manufacturing techniques known in the art.

A second end 138 of each fiber 126 is mounted to a mounting plate 140 so that when the mounting plate is attached (optionally using a suitable adhesive 26) to a region of skin 22, the second ends of the fibers are in optical and acoustic contact with the skin. Optionally, each second end 138 is formed with a lens (not shown) and/or coupled to optics (not shown) formed in mounting plate 140 that shapes light from light source 134 that exits the second end into a beam of light (not shown) having a desired shape. Optionally, the lens and/or optics shapes the light into a cone beam. In some embodiments of the invention the lens and/or optics shapes the light into a fan beam. Each optical fiber 126 functions not only to transmit light from its associated light source 134 to illuminate tissue below skin 22. It also functions to propagate acoustic energy that reaches its end 138 from photoacoustic waves stimulated in the tissue by the light to its associated acoustic transducer 132.

A controller 46 controls light sources 134 and receives signals generated by transducers 132 responsive to acoustic energy that the transducers receive via optic fibers 126. Optionally controller 46 is configured to control transducers 132 to transmit ultrasound into tissue below skin 22 via optic fibers 126 for situations in which it is advantageous to acoustically image features in the tissue.

Components of glucometer 120 are contained in a housing 150 shown in dashed lines. Optionally, a power source 45 for powering light sources 134 and controller 45 is mounted inside the housing. In some embodiments of the invention glucometer 120 receives power from an external power source optionally mounted to the patient's body. Housing 150 optionally comprises a visual display screen and control buttons (not shown) for transmitting commands and or data to controller 46.

Whereas in glucometer 120 each fiber 126 functions to transmit light and acoustic energy and is mounted to both an acoustic transducer 132 and to a light source 134, in some embodiments of the invention, optic fibers in a glucometer similar to glucometer 120 are not coupled to both an acoustic transducer and a light source. Instead, each acoustic transducer 132 is mounted to an acoustic waveguide, which may be an optic fiber, which is not coupled to a light source 134 and each light source 134 is mounted to an optic fiber 126, which is not mounted to an acoustic transducer 132.

In some embodiments of the invention, a glucometer similar to a glucometer described herein is used not only to monitor a patient's blood glucose but also to control the patient's blood glucose. The glucometer is connected to a suitable insulin delivery system, such as for example, an insulin pump coupled to a needle or a drug delivery patch that is controllable to administer insulin to a patient. The glucometer and delivery system are mounted to the patient's body. The glucometer controller controls the delivery system to administer insulin to the patient and control thereby the patient's blood glucose level responsive to blood glucose measurements provided by the glucometer.

It is noted that whereas the glucometers discussed above are described as being used to assay glucose, the glucometers may be used to assay an analyte in blood in a blood vessel other than glucose. To assay an analyte in a blood vessel other than glucose, a glucometer in accordance with an embodiment of the invention is operated similarly to the way in which it is operated to assay glucose but with the glucometer's light provider providing light that is absorbed and/or scattered by the other analyte.

In the description and claims of the present application, each of the verbs, “comprise” “include” and “have”, and conjugates thereof, are used to indicate that the object or objects of the verb are not necessarily a complete listing of members, components, elements or parts of the subject or subjects of the verb.

The present invention has been described using detailed descriptions of embodiments thereof that are provided by way of example and are not intended to limit the scope of the invention. The described embodiments comprise different features, not all of which are required in all embodiments of the invention. Some embodiments of the present invention utilize only some of the features or possible combinations of the features. Variations of embodiments of the present invention that are described and embodiments of the present invention comprising different combinations of features noted in the described embodiments will occur to persons of the art. The scope of the invention is limited only by the following claims.

1. Apparatus for assaying an analyte in blood in a patient's blood vessel comprising:
a light provider comprising at least one light source that illuminates a tissue region in which a blood vessel is located with light that stimulates photoacoustic waves in the region;
at least one acoustic transducer that generates signals responsive to the photoacoustic waves;
a controller that receives the signals and processes them to determine which are responsive to photoacoustic waves that originate in the blood vessel and uses the determined signals to assay the analyte; wherein,
the light provider and at least one transducer define a field of view that overlaps the blood vessel, said field of view having a central region and a lateral extent greater than about 4 mm.

2. Apparatus according to claim 1 wherein the field of view has a lateral extent greater than or equal to about 6 mm.

3. Apparatus according to claim 1 wherein the field of view has a lateral extent greater than or equal to about 10 mm.

4. Apparatus according to any of claims 1-3 wherein the light provider comprises optics for each of the at least one light source that receives light from the light source and configures the received light into a fan shaped light beam that is used to illuminate the tissue region.

5. Apparatus according to claim 4 wherein the at least one light source comprises a plurality of light sources.

6. Apparatus according to claim 5 wherein the fan beams of the plurality of light sources are substantially parallel.

7. Apparatus according to claim 6 wherein the plurality of light sources are collinear.

8. Apparatus according to claim 6 wherein the plurality of light sources are configured in an array of rows and columns.

9. Apparatus according to any of claims 1-8 wherein the light provider comprises a mirror that receives light from the light source and reflects the received light to the tissue region and wherein the mirror is rotatable about an axis and for different rotation angles of the mirror about the axis the fan beam illuminates a different portion of the tissue region.

10. Apparatus according to claim 9 and comprising a controller that controls the angle of the mirror to scan the tissue region with light from the light source.

11. Apparatus according to any of the preceding claims wherein the light provider comprises a light pipe having an input surface region to which at least one light source is coupled and an output surface region through which light that enters the light pipe from the at least one light source exits the light pipe.

12. Apparatus according to claim 11 wherein the light pipe has a shape of a planar plate having two large parallel face surfaces and narrow edge surfaces.

13. Apparatus according to claim 12 wherein the input surface region to which the at least one light source is coupled is a narrow edge surface of the light pipe.

14. Apparatus according to claim 13 wherein the output surface region from which light exits the light pipe is a narrow edge surface opposite the input surface region.

15. Apparatus according to any of the preceding claims wherein the at least one transducer comprises a plurality of transducers.

16. Apparatus according to claim 15 wherein the transducers are configured in an array of rows and columns of transducers.

17. Apparatus according to claim 15 or claim 16 and comprising a mounting plate, which is attached to the skin to acoustically couple the apparatus to the skin.

18. Apparatus according to claim 17 and wherein the transducers are mounted to the mounting plate.

19. Apparatus according to claim 17 wherein the mounting plate comprises a layer of piezoelectric material.

20. Apparatus according to claim 19 wherein each of at least two of the plurality of transducers comprises a different region of the layer of piezoelectric material sandwiched between a first and a second electrode.

21. Apparatus according to claim 20 wherein the first electrodes of each of the at least two transducers are substantially electrically isolated from each other.

22. Apparatus according to claim 21 wherein the second electrode of each of the at least two transducers comprises a different region of a same conductor.

23. Apparatus according to any of claims 1-22 wherein a transducer of the at least one transducer is acoustically coupled to the skin via an acoustic waveguide.

24. Apparatus according to claim 23 wherein the acoustic waveguide is an optic fiber.

25. Apparatus according to any of claims 1-24 wherein a light source of the at least one light source is optically coupled to the skin via an optic fiber that transmits light from the light source to the skin.

26. Apparatus according to claim 25 wherein a transducer of the at least one transducer light is acoustically coupled to the skin by the optic fiber.

27. Apparatus according to any of the preceding claims wherein the controller controls the at least one transducer to acoustically image the blood vessel.

28. Apparatus according to any of the preceding claims wherein the controller processes signals generated by the at least one transducer responsive to acoustic energy from the photoacoustic waves to image the blood vessel.

29. Apparatus according to claim 28 wherein at least some of the light provided by the light provider is light at a wavelength at which light is strongly absorbed and or scattered by blood.

30. Apparatus according to any of claims 27-29 wherein the controller uses the image to determine if the blood vessel is substantially aligned with the central region of the field of view.

31. Apparatus according to claim 30 wherein the apparatus comprises an indicator light and the controller controls the indicator light to generate an optical signal indicative of a degree to which the blood vessel is aligned with the central region.

32. Apparatus according to claim 30 or claim 31 wherein the apparatus comprises a speaker and the controller controls the speaker to generate an audio signal indicative of a degree to which the blood vessel is aligned with the central region.

33. Apparatus according to any of claims 27-32 wherein the apparatus comprises a display screen and the controller displays a fiducial mark representing the central region of the field of view and the image of the blood vessel on the screen and wherein a distance on the screen between the blood vessel and the fiducial mark represents a distance between the blood vessel and the central region.

34. Apparatus according to any of the preceding claims wherein the light provider and at least one transducer are comprised in a wearable housing.

35. Apparatus according to claim 34 wherein when worn by the patient the housing provides optical and acoustic coupling of the light provider and at least one transducer respectively to the patient's skin.

36. Apparatus according to any of the preceding claims wherein the analyte is glucose.

37. Apparatus for controlling blood glucose level in a patient comprising:
assay apparatus according to claim 36;
an insulin delivery system controllable to administer insulin to a patient;
wherein the controller controls the insulin delivery system responsive to glucose assays provided by the assay apparatus.
